(19) United States
(12) Patent Application Publication
(10) Pub. No.: US 2002/0091254 A1
(43) Pub. Date: Jul. 11, 2002

(54) Stabilizing and/or lowering the color number of alkenyl compounds

(76) Inventors: Rudolf Erich Lorenz, Ludwigshafen (DE); Arnd Bottcher, Frankenthal (DE); Heike Becker, Frankenthal (DE); Rolf Pinkos, Bad Durkheim (DE)

Correspondence Address: KEIL & WEINKAUF, 1350 CONNECTICUT AVENUE, N.W., WASHINGTON, DC, 20036, US

(21) Appl. No.: 10/033,911

(22) Filed: Jan. 3, 2002

(30) Foreign Application Priority Data
Jan. 10, 2001 (DE) 10100751.5

(51) Int. Cl. C07C 211/00; C07D 223/10

(52) U.S. Cl. 540/485; 564/1

(57) ABSTRACT

A process is provided for stabilizing and/or lowering the color number of alkenyl compounds containing a divalent or trivalent heteroatom in the α-position to the double bond, wherein an oxidizing agent is added to the alkenyl compounds.

The present invention relates to a process for stabilizing and/or lowering the color number of alkenyl compounds containing a divalent or trivalent heteroatom in the α-position to the double bond, especially alkenyl compounds of general formula (Ia) or (Ib):

R1−X−CR4=CHR5 (1a)

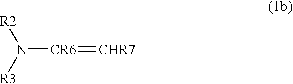

in which X is a divalent heteroatom, R¹, R² and R³ independently of one another are each a carbon-containing organic radical, it also being possible for R² and R³ to be linked together, and R⁴, R⁵, R⁶ and R⁷ independently of one another are each hydrogen or a hydrocarbon radical.

Alkenyl compounds are used inter alia as monomeric structural units in oligomers, polymers and copolymers. Thus alkenyl compounds find their way into the manufacture of e.g. paper coatings, adhesives, printing inks, detergents, engine oil additives, textile auxiliaries, radiation-curing surface coatings, cosmetics, pharmaceuticals, auxiliaries for petroleum production or chemicals for photographic applications.

Alkenyl compounds are obtained industrially by a variety of processes, for example by addition onto alkynes (alkenylation), transfer of alkenyl groups, elimination to form the double bond, or oxidative addition onto alkenes. A survey of the preparation of vinyl ethers and vinyl esters can be found in Ullmann's Encyclopedia of Industrial Chemistry, 6^{th} edition, 1999 Electronic Release, Chapter “VINYL ETHERS” and Chapter “VINYL ESTERS”.

W. Reppe et al., Justus Liebigs Ann. Chem., vol. 601, 1956, pages 81 to 138, describes the preparation of vinyl ethers, vinyl esters, vinylamines, vinyl-N-heterocycles and vinylamides by reacting ethyne with the appropriate alcohols, carboxylic acids, amines, NH-heterocycles and amides in the presence of basic catalysts.

As is apparent from the documents cited, the actual synthesis step is conventionally followed by a distillative purification, in which the desired products can be obtained in high purity by condensation from the gas phase. Purities of well over 99% can thus be achieved without problems for very many alkenyl compounds, which is also totally satisfactory for a large number of applications. To suppress unwanted reactions such as decomposition, oligomerization or polymerization during storage and transportation, a stabilizer is conventionally added. A frequently used stabilizer is N,N′-bis(1-methylpropyl)-1,4-phenylenediamine, which is marketed by BASF AG under the trade name Kerobit® BPD. Examples of other known stabilizers are alkali metal hydroxides or phenothiazine derivatives.

For applications where the inherent color of the product should be minimal, for example in the cosmetic or photographic sector or in paper coatings, there is a need not only for a high chemical purity but also for a very high purity in respect of color-causing impurities. A few ppb by weight of color-causing impurities are generally sufficient to discolor the product substantially. Expensive purification processes, for example multiple distillation or crystallization, are conventionally required in order to obtain alkenyl compounds with a very low color number.

Alkenyl compounds normally exhibit a tendency to discolor both in the presence and in the absence of a stabilizer. As a result the product has a markedly darker color after storage or transportation than before. This disadvantageous behavior thus has a decisive influence on the product quality with the practical consequence that either a poorer product quality has to be accepted or another expensive purification has to be carried out before the alkenyl compounds are used. It is an object of the present invention to find a process for stabilizing and/or lowering the color number of alkenyl compounds which no longer has the abovementioned disadvantages and produces alkenyl compounds with a very low and stabilized color number without great expense, said alkenyl compounds exhibiting no tendency or only a very low tendency to discolor, even after prolonged storage for several months.

Surprisingly, we have found that this object is achieved by a process for stabilizing and/or lowering the color number of alkenyl compounds containing a divalent or trivalent heteroatom in the α-position to the double bond, wherein an oxidizing agent is added to the alkenyl compounds.

The term “oxidizing agents” is to be understood as meaning elements and compounds which endeavor, by taking up electrons as a result of chemical interaction with a reactant, to pass to a lower-energy state with the formation of stable electron shells (cf. CD-Römpp Chemie Lexikon, “Oxidantien”, version 1, Stuttgart/New York, Georg Thieme Verlag 1995). The oxidizing agents are reduced in this process.

The oxidizing agents which can be used in the process according to the invention can be of a gaseous, liquid or solid nature. For example, they can have a neutral charge or be in ionic or zwitterionic form. The oxidizing agents can also be elements or compounds. Preferred oxidizing agents are those which have only a very weak inherent color or are colorless.

Examples which may be mentioned of oxidizing agents having a neutral charge are molecular oxygen (O₂), ozone (O₃), chlorine (Cl₂), halogen oxides (e.g. chlorine dioxide), hypohalous acids (e.g. hypochlorous acid), halous acids (e.g. chlorous acid), halic acids (e.g. chloric acid), perhalic acids (e.g. perchloric acid) and peroxides such as hydrogen peroxide, hydroperoxides, “peroxides” (R-O—O-R), diacyl peroxides, per acids, per acid esters, ketone peroxides and epidioxides. Examples of ionic oxidizing agents which may be mentioned are peroxodisulfates (e.g. sodium or potassium peroxodisulfate), hypohalites (e.g. sodium or potassium hypochlorite), halites (e.g. sodium or potassium chlorite), halogenates (e.g. sodium or potassium chlorate), perhalogenates (e.g. sodium or potassium perchlorate) or metal peroxides (e.g. sodium peroxide or barium peroxide).

The process according to the invention is carried out using preferably an oxygen-transferring oxidizing agent and particularly preferably molecular oxygen (O₂), ozone (O₃), peroxides or mixtures thereof. The following may be mentioned as particularly preferred peroxides:

hydrogen peroxide,

hydroperoxides, e.g. tert-butyl hydroperoxide or cumene hydroperoxide,

“peroxides” (R-O—O-R), e.g. ditert-butyl peroxide,

diacyl peroxides, e.g. dibenzoyl peroxide,

per acids, e.g. perbenzoic acid or benzoylpercarbamic acid, and

per acid esters, e.g. tert-butyl perbenzoate.

The use of molecular oxygen (O₂), hydrogen peroxide and tert-butyl hydroperoxide is very particularly preferred.

The oxidizing agents can be added undiluted or else diluted with other gases, liquids (e.g. solvents) or solids. Thus, for example, molecular oxygen (O₂) can be added as pure oxygen gas or diluted with other gases, for instance nitrogen, noble gases (e.g. argon, helium, neon), carbon dioxide or water vapor. When adding molecular oxygen, it is particularly preferred to use air. The ozone (O₃) suitable for addition can be obtained for example by the ozonization of molecular oxygen, especially pure oxygen or air.

The addition of the oxidizing agents results in a stabilization and/or lowering of the color number of the alkenyl compound. Said color number is a characteristic of the color of transparent compounds. The lower the color number, the more colorless the product is. APHA, which is defined in DIN EN 1557 (March 1997), is a widely used method of determining the color number.

The oxidizing agents to be used in the process according to the invention can be e.g. chemically or physically dissolved, emulsified or suspended in the alkenyl compounds.

The amount of oxidizing agent to be used normally depends on the nature and amount of the color-causing impurities, the desired stabilizing effect or the desired lowering of the color number, and can be adapted to the system in question by means of simple experiments. The content of dissolved oxidizing agent is generally adjusted to 0.0001 to 1000 ppm by weight and preferably to 0.001 to 500 ppm by weight, based on the alkenyl compound.

The process according to the invention is generally carried out at a temperature of 0° to 100° C. and preferably of 10° to 70° C., the alkenyl compound preferably being in the liquid phase. It is generally carried out at a pressure of 0.01 to 100 MPa abs, preferably of 0.05 to 10 MPa abs and especially under atmospheric pressure.

When using liquid or solid oxidizing agents and mixtures or solutions thereof, the general procedure is to add the desired amount to the preferably liquid alkenyl compound, with mixing. When using gaseous oxidizing agents, the general procedure is to add them by introducing them directly into or simply bringing them into contact with the preferably liquid alkenyl compound. The components can be brought into contact with one another for example by simply covering the preferably liquid alkenyl compound with a layer of the gaseous oxidizing agent or by specifically introducing the gaseous oxidizing agent into the preferably liquid alkenyl compound.

If it is desired specifically to lower the color number, the oxidizing agent is generally allowed to act for a few minutes to a few days. The required time depends inter alia on the nature of the color-causing impurities, the nature and concentration of the oxidizing agent and the desired color number of the product treated. If gaseous oxidizing agents, for example molecular (O₂), are used, they are advantageously added continuously or periodically throughout the period. Liquid or solid oxidizing agents are normally added at the start, although further additions at a later stage are of course also possible.

If it is desired primarily to stabilize the color number, the oxidizing agent is generally allowed to act for a longer period of days, weeks, months or years. Thus the alkenyl compound can also be stored or transported over a longer period. As is generally known, the color number of alkenyl compounds increases over time if the measure according to the invention is not taken. A stabilization of the color number is to be understood in terms of the present invention as meaning a development of the color number of the alkenyl compound which leads to lower values than when the measure according to the invention is not taken. Thus, when the color number is stabilized, it can (i) increase more slowly than when the measure according to the invention is not taken, (ii) remain almost unchanged, or (iii) decrease over time. For stabilization of the color number, the oxidizing agent advantageously acts over the entire period. If gaseous oxidizing agents, for example molecular oxygen (O₂), are used, they are preferably introduced into the product by being passed through the preferably liquid alkenyl compounds or by covering them with a blanket of gas.

Stabilizers can be added to the alkenyl compounds used in the process according to the invention in order to suppress unwanted reactions such as decomposition, oligomerization or polymerization. Examples of suitable stabilizers which may be mentioned are N,N′-bis(1-methylpropyl)-1,4-phenylenediamine, which is marketed by BASF AG under the trade name Kerobit® BPD, alkali metal hydroxides or phenothiazine derivatives. If stabilizers are added to the alkenyl compounds, their content is generally 1 to 1000 ppm by weight, preferably 1 to 100 ppm by weight and particularly preferably 5 to 50 ppm by weight.

The alkenyl compounds to be used in the process according to the invention have e.g. general formula (Ia) or (Ib):

R1−X−CR4=CHR5 (1a)

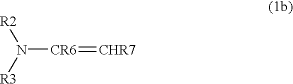

in which X is a divalent heteroatom, R¹, R² and R³ independently of one another are each a carbon-containing organic radical, it also being possible for R² and R³ to be linked together, and R⁴, R⁵, R⁶ and R⁷ independently of one another are each hydrogen or a hydrocarbon radical.

A carbon-containing organic radical is to be understood as meaning an unsubstituted or substituted aliphatic, aromatic or araliphatic radical having from 1 to 22 carbon atoms. This radical can contain one or more heteroatoms such as oxygen, nitrogen or sulfur, for example —O—, —S—, —NR—, —CO— and/or N═ in aliphatic or aromatic systems, and/or can be substituted by one or more functional groups containing e.g. oxygen, nitrogen, sulfur and/or halogen, for example by fluorine, chlorine, bromine, iodine and/or a cyano group. If the carbon-containing organic radical contains one or more heteroatoms, it can also be bonded via a heteroatom or a carbon atom carrying a heteroatom. Thus, for example, radicals bonded via a nitrogen atom or a CO group are also included.

The following may be mentioned as preferred monovalent (i.e. terminal) carbon-containing organic radicals for R¹, R² and R³:

unbranched or branched, acyclic and cyclic alkyl having from 1 to 22 aliphatic carbon atoms, in which one or more of the —CH₂— groups can also be replaced with heteroatoms such as O—, or by heteroatom-containing groups such as CO— or NR—, and in which one or more of the hydrogen atoms can be replaced with substituents such as aryl groups,

unbranched or branched, acyclic and cyclic alkenyl having from 2 to 22 aliphatic carbon atoms and one or more double bonds in any position, in which one or more of the —CH₂— groups can also be replaced with heteroatoms such as O—, or by heteroatom-containing groups such as CO— or NR—, and in which one or more of the hydrogen atoms can be replaced with substituents such as aryl groups,

aryl having up to 10 aromatic carbon atoms, in which one or more of the ═CH— groups can be replaced with heteroatoms such as ═N—, and in which one or more of the hydrogen atoms can be replaced with substituents such as alkyl groups,

and radicals as mentioned above in which one or more of the hydrogen atoms are substituted by a X-CR⁴═CHR⁵ or >Y-CR⁶═CHR⁷— group.

The following may be mentioned as preferred divalent (i.e. linked) carbon-containing organic radicals for R²-R³:

unbranched or branched alkylene having from 3 to 20 aliphatic carbon atoms, in which one or more of the —CH₂— groups can also be replaced with heteroatoms such as O—, or by heteroatom-containing groups such as CO— or NR—, and in which one or more of the hydrogen atoms can be replaced with substituents such as aryl groups,

and unbranched or branched alkenylene having from 3 to 20 carbon atoms and one or more double bonds, in which one or more of the —CH₂— groups can also be replaced with heteroatoms such as O—, or with heteroatom-containing groups such as CO— or NR—, in which furthermore one or more of the ═CH— groups can be replaced with heteroatoms such as ═N—, and in which one or more of the hydrogen atoms can be replaced with substituents such as aryl groups.

The divalent heteroatom X mentioned in the alkenyl compounds (Ia) can be an oxygen atom or a sulfur atom. Examples of alkenyl compounds (Ia) which may be mentioned are alkenyl ethers, alkenyl esters and alkenyl sulfides. The process according to the invention is preferably carried out using alkenyl compounds (Ia) in which X is oxygen.

Examples of alkenyl compounds (Ib) which may be mentioned are alkenylamines, N-alkenylamides and N-alkenylheterocycles. N-Alkenylamides include cyclic N-alkenylamides, also known as N-alkenyllactams.

A hydrocarbon radical is to be understood as meaning an aliphatic, aromatic or araliphatic radical having from 1 to 12 carbon atoms. Preferred hydrocarbon radicals which may be mentioned for R⁴, R⁵, R⁶ and R⁷ are C₁- to C₄-alkyl, for example methyl, ethyl, 1-propyl, 2-propyl, 1-butyl, 2-butyl, 2-methyl-1-propyl and 2-methyl-2-propyl, especially methyl, C₆-aryl, phenyl itself, C₇- to C₈-aralkyl, for example phenylmethyl and phenylethyl, and C₇- to C₈-alkaryl, for example 2-methylphenyl, 3-methylphenyl and 4-methylphenyl.

Particularly preferred alkenyl compounds (Ia) and (Ib) are those in which the radicals R⁴, R⁵, R⁶ and R⁷ independently of one another are hydrogen or methyl. Very particularly preferred alkenyl compounds (Ia) and (Ib) are those in which the radicals R⁴, R⁵, R⁶ and R⁷ are hydrogen, i.e. vinyl compounds.

Examples which may be mentioned of the alkenyl sulfides of formula (Ia) where X is sulfur, which can be used in the process according to the invention, are vinyl methyl sulfide, vinyl ethyl sulfide, vinyl (1-propyl) sulfide, vinyl (2-propyl) sulfide (vinyl isopropyl sulfide), vinyl (1-butyl) sulfide, vinyl (2-butyl) sulfide (vinyl sec-butyl sulfide), vinyl (2-methyl-2-propyl) sulfide (vinyl tert-butyl sulfide), vinyl pentyl sulfide and isomers thereof, and vinyl hexyl sulfide and isomers thereof.

The alkenyl compounds (Ia) in the process according to the invention are particularly preferably vinyl ethers. Examples of preferred vinyl ethers which may be mentioned are vinyl methyl ether, vinyl ethyl ether, vinyl (1-propyl) ether, vinyl (2-propyl) ether (vinyl isopropyl ether), vinyl (1-butyl) ether, vinyl (2-butyl) ether (vinyl sec-butyl ether), vinyl (2-methyl-2-propyl) ether (vinyl tert-butyl ether), vinyl pentyl ether and isomers thereof, vinyl hexyl ether and isomers thereof, vinyl heptyl ether and isomers thereof, vinyl octyl ether and isomers thereof, vinyl nonyl ether and isomers thereof, vinyl decyl ether and isomers thereof, vinyl undecyl ether and isomers thereof, vinyl dodecyl ether and isomers thereof, vinyl tridecyl ether and isomers thereof, vinyl tetradecyl ether and isomers thereof, vinyl pentadecyl ether and isomers thereof, vinyl hexadecyl ether and isomers thereof, vinyl heptadecyl ether and isomers thereof, vinyl octadecyl ether and isomers thereof, vinyl nonadecyl ether and isomers thereof, vinyl eicosyl ether and isomers thereof, vinyl heneicosyl ether and isomers thereof, vinyl docosyl ether and isomers thereof, vinyl cyclopentyl ether, vinyl cyclohexyl ether, vinyl cycloheptyl ether, vinyl cyclooctyl ether, vinyl cyclododecyl ether, vinyl phenyl ether, vinyl (2-methylphenyl) ether, vinyl (3-methylphenyl) ether, vinyl (4-methylphenyl) ether, vinyl (phenylmethyl) ether, vinyl (2-phenylethyl) ether, 2-hydroxyethyl 1-vinyl ether (3-oxapenta-4-en-1-ol), ethylene glycol divinyl ether (3,6-dioxaocta-1,7-diene), diethylene glycol monovinyl ether (3,6-dioxaocta-7-en-1-ol), diethylene glycol divinyl ether (3,6,9-trioxaundeca-1,10-diene), triethylene glycol monovinyl ether (3,6,9-trioxaundeca-10-en-1-ol), triethylene glycol divinyl ether (3,6,9,12-tetraoxatetradeca-1,13-diene), tetraethylene glycol monovinyl ether (3,6,9,12-tetraoxatetradeca-13-en-1-ol), tetraethylene glycol divinyl ether (3,6,9,12,15-pentaoxaheptadeca-1,16-diene), 1,2-propylene glycol monovinyl ether (4-oxahexa-5-en-2-ol and 2-methyl-3-oxapenta-4-en-1-ol), 1,2-propylene glycol divinyl ether (4-methyl-3,6-dioxaocta-1,7-diene), 3-hydroxypropyl 1-vinyl ether (5-oxahepta-6-en-1-ol), 1,3-propylene glycol divinyl ether (3,7-dioxanona-1,8-diene), 4-hydroxybutyl 1-vinyl ether (5-oxahepta-6-en-1-ol), 1,4-butylene glycol divinyl ether (3,8-dioxadeca-1,9-diene), 5-hydroxypentyl 1-vinyl ether (6-oxaocta-7-en-1-ol), 1,5-pentylene glycol divinyl ether (3,9-dioxaundeca-1,10-diene), 6-hydroxyhexyl 1-vinyl ether (7-oxanona-8-en-1-ol), 1,6-hexylene glycol divinyl ether (3,10-dioxadodeca-1,11-diene), 8-hydroxyoctyl 1-vinyl ether (9-oxaundeca-10-en-1-ol), 1,8-octylene glycol divinyl ether (3,12-dioxatetradeca-1,13-diene), 12-hydroxydodecyl 1-vinyl ether (13-oxapentadeca-14-en-1-ol), 1,12-dodecylene glycol divinyl ether (3,16-dioxaoctadeca-1,17-diene), 4-hydroxycyclohexyl 1-vinyl ether, 1,4-cyclohexylene divinyl ether (1,4-bis(vinyloxy)cyclohexane), 4-vinyloxyphenol and bis(vinyloxy)-1,4-phenylene.

Very particularly preferred vinyl ethers in the process according to the invention are ethylene glycol divinyl ether (3,6-dioxaocta-1,7-diene), diethylene glycol divinyl ether (3,6,9-trioxaundeca-1,10-diene), triethylene glycol divinyl ether (3,6,9,12-tetraoxatetradeca-1,13-diene) and 4-hydroxybutyl 1-vinyl ether (5-oxahepta-6-en-1-ol).

The alkenyl compounds (Ib) in the process according to the invention are particularly preferably acyclic and cyclic N-vinylamines, acyclic and cyclic N-vinylamides and N-vinylheterocycles, especially N-vinylamides and N-vinylheterocycles.

Examples which may be mentioned of preferred acyclic and cyclic N-vinylamines are N-vinyldimethylamine, N-vinyldiethylamine, N-vinyldi(1-propyl)amine, N-vinyldi(2-propyl)amine (N-vinyldiisopropylamine), N-vinyldi(1-butyl)amine, N-vinyldi(2-butyl)amine (N-vinyldisec-butylamine), N-vinyldi(2-methyl-2-propyl)amine (N-vinylditert-butylamine), N-vinylmethylethylamine, N-vinylmethyl(1-propyl)amine, N-vinylmethyl(2-propyl)amine (N-vinylmethylisopropylamine), N-vinylmethyl(1-butyl)amine, N-vinylmethyl(2-butyl)amine (N-vinylmethylsec-butylamine), N-vinylmethyl(2-methyl-2-propyl)amine (N-vinylmethyl-tert-butylamine), N-vinylmethylpentylamine and isomers thereof, N-vinylmethylhexylamine and isomers thereof, N-vinylmethylheptylamine and isomers thereof, N-vinylmethyloctylamine and isomers thereof, N-vinylmethylnonylamine and isomers thereof, N-vinylmethyldecylamine and isomers thereof, N-vinylmethylundecylamine and isomers thereof, N-vinylmethyldodecylamine and isomers thereof, N-vinylmethyltridecylamine and isomers thereof, N-vinylmethyltetradecylamine and isomers thereof, N-vinylmethylpentadecylamine and isomers thereof, N-vinylmethylhexadecylamine and isomers thereof, N-vinylmethylheptadecylamine and isomers thereof, N-vinylmethyloctadecylamine and isomers thereof, N-vinylmethylnonadecylamine and isomers thereof, N-vinylmethyleicosylamine and isomers thereof, N-vinylmethylheneicosylamine and isomers thereof, N-vinylmethyldocosylamine and isomers thereof, N-vinylmethylcyclopentylamine, N-vinylmethylcyclohexylamine, N-vinylmethylcycloheptylamine, N-vinylmethylcyclooctylamine, N-vinylmethylcyclododecylamine, N-vinylmethylphenylamine, N-vinyldiphenylamine, N-vinylmethyl(2-methylphenyl)amine, N-vinylmethyl(3-methylphenyl)amine, N-vinylmethyl(4-methylphenyl)amine, N-vinylmethyl(phenylmethyl)amine, N-vinylmethyl(2-phenylethyl)amine, N-vinylpyrrolidine, N-vinylpiperidine and N-vinylmorpholine.

Examples which may be mentioned of preferred acyclic and cyclic N-vinylamides are N-vinyl-N-methylacetamide, N-vinylpyrrolidone, N-vinyl-2-piperidone (N-vinyl-δ-valerolactam), N-vinyl-ε-caprolactam (N-vinyl-6-aminohexanoic acid lactam), N-vinyl-7-aminoheptanoic acid lactam, N-vinyl-8-aminooctanoic acid lactam, N-vinyl-9-aminononanoic acid lactam, N-vinyl-10-aminodecanoic acid lactam, N-vinyl-12-aminododecanoic acid lactam (N-vinyllaurolactam).

Examples of preferred N-vinylheterocycles which may be mentioned are N-vinylpyrrole, N-vinylpyrazole, N-vinylimidazole, N-vinyl-1,2,3-triazole, N-vinyl-1,2,4-triazole, N-vinyl-1,3,4-triazole and N-vinyl-2-methylimidazole, especially N-vinylimidazole.

It is very particularly preferred to use N-vinyl-ε-caprolactam in the process according to the invention, it being possible for the latter to be in the solid phase, in the liquid phase or else in a mixture of the two phases. The N-vinyl-ε-caprolactam is preferably kept in the liquid phase, particularly preferably over 90% by weight, very particularly preferably over 99% by weight and especially the whole of the N-vinyl-ε-caprolactam being in the liquid phase.

To keep the N-vinyl-ε-caprolactam in the liquid phase, the temperature used generally corresponds to the melting point or is above the melting point. Pure N-vinyl-ε-caprolactam has a melting point of 35° C. In the process according to the invention, the N-vinyl-ε-caprolactam is preferably kept at a temperature of 35° to 100° C., particulary preferably of 35° to 75° C. and very particularly preferably of 35° to 60° C.

In one embodiment for lowering the color number, air is passed through the liquid alkenyl compound for a period of several minutes to a few days. When the desired lower color number is reached, the introduction of air is stopped and the product can be processed further or stored, for example.

In one embodiment for stabilizing the color number, the alkenyl compound is transferred to a container and the product is covered with a layer of air. The alkenyl compound can then be stored or transported in the solid or liquid state. In another embodiment for stabilizing the color number, a solid or liquid oxidizing agent is dissolved in the liquid alkenyl compound, it then being possible for the latter to be stored or transported in the solid or liquid state.

In one preferred embodiment for stabilizing the color number of N-vinyl-ε-caprolactam, the liquid product is transferred to containers, covered with a layer of air and stored or transported at 35° to 60° C.

In another preferred embodiment for stabilizing the color number of N-vinyl-ε-caprolactam, a solid or liquid oxidizing agent (e.g. a peroxide) is dissolved in the liquid product and the latter is stored or transported in the liquid state at 35° to 60° C.

The process according to the invention for stabilizing and/or lowering the color number of alkenyl compounds is particularly surprising because said compounds are sensitive to polymerization and, as is generally known to those skilled in the art, oxidizing agents can be expected to cause unwanted and uncontrolled secondary reactions such as oligomerization or polymerization. Those skilled in the art would therefore generally expect the color number to increase. It is completely unexpected to observe the opposite effect, namely a lowering of the color number.

The process according to the invention makes it possible to stabilize and/or lower the color number of alkenyl compounds without great expense to give alkenyl compounds with a very low and stabilized color number which exhibit no tendency to discolor, even after prolonged storage for several months.

### EXAMPLES

To characterize the possible discoloration of the alkenyl compounds, the color numbers were determined by the APHA method, analogously to DIN EN 1557.

### Examples 1 and 2

The N-vinyl-ε-caprolactam used in Examples 1 to 6 had a purity of 99.7% by weight of N-vinyl-ε-caprolactam and was stabilized with about 10 ppm by weight of N,N′-bis(1-methylpropyl)-1,4-phenylenediamine (trade name Kerobit® BPD). The content of residual ε-caprolactam was about 0.3% by weight.

In Comparative Example 1, approx. 900 g of liquid N-vinyl-ε-caprolactam with an APHA color number of 80 were transferred under a protective nitrogen atmosphere to a nitrogen-filled 1000 ml polyethylene bottle, and the bottle was sealed. The N-vinyl-ε-caprolactam was then stored as a supercooled melt at about 25° C. with the blanket of nitrogen on top. The APHA color number was determined again after nine months. It was 238.

In Example 2 according to the invention, approx. 900 g of liquid N-vinyl-ε-caprolactam with an APHA color number of 80 were transferred to an air-filled 1000 ml polyethylene bottle, and the bottle was sealed. The N-vinyl-ε-caprolactam was then stored as a supercooled melt at about 25° C. with the blanket of air on top. The bottle was opened for approx. 30 seconds every month in order to renew the blanket of air. The APHA color number was determined again after nine months. It was 19.

Comparative Example 1 shows that, without the addition of an oxidizing agent, N-vinyl-ε-caprolactam has a very pronounced tendency to discolor, the APHA color number rising significantly from 80 to 238 within nine months. In the presence of an oxidizing agent, on the other hand, the color number dropped markedly from 80 to 19, as shown by Example 2 according to the invention. Thus the color number in Example 2 according to the invention was only about 8% of the color number in Comparative Example 1. Example 2 confirms both a marked lowering and a pronounced stabilization of the color number.

### Example 3

The N-vinyl-ε-caprolactam from Comparative Example 1, with an APHA color number of 238, was heated to 40° C. and air was bubbled through it via an inlet tube for four days. The color number dropped to 183 during this time, corresponding to a lowering of the value by about 1/4.

### Example 4

500 g of N-vinyl-ε-caprolactam (unstabilized, APHA color number of 45) were stored for 95 hours at 40° C. During this time a stream of air was passed through the product. The color number was 22 at the end of the experiment.

### Example 5

500 g of N-vinyl-ε-caprolactam (stabilized with 10 ppm by weight of Kerobit, APHA color number of 45) were stored for 71 hours at 40° C. A stream of nitrogen was passed through the product during the first 69 hours, the color number rising to 75. Air was then passed through for a further 2 hours, the color number dropping to 64.

### Example 6

5 g of N-vinyl-ε-caprolactam (stabilized with 10 ppm by weight of Kerobit, APHA color number of 174) were treated with 50 ppm by weight of 30% aqueous hydrogen peroxide solution and stored at 50° C. for 3 days, after which the measured APHA color number was 101.

### Example 7

A 220 1 drum containing 190 kg of diethylene glycol divinyl ether (covered with nitrogen, APHA color number of 667) was opened at the bunghole (aperture approx. 10 cm) and stored in the air for 11 days, the APHA color number dropping to 425.

We claim:

1. A process for stabilizing and/or lowering the color number of alkenyl compounds containing a divalent or trivalent heteroatom in the a-position to the double bond, wherein an oxidizing agent is added to the alkenyl compounds.

2. A process as claimed in claim 1 wherein the oxidizing agent used is an oxygen-transferring oxidizing agent.

3. The process as claimed in claim 2 wherein the oxygen-transferring oxidizing agent used is molecular oxygen, ozone, hydrogen peroxide, organic peroxides or mixtures thereof.

4. The process as claimed in claim 1 wherein the content of dissolved oxidizing agent is adjusted to 0.0001 to 1000 ppm by weight, based on the alkenyl compound.

5. The process as claimed in claim 1 wherein the alkenyl compounds are compounds of general formula (Ia) or (Ib):
R1−X−CR4=CHR5 (1a) 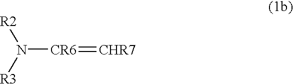
in which X is a divalent heteroatom, R¹, R² and R³ independently of one another are each a carbon-containing organic radical, it also being possible for R² and R³ to be linked together, and R⁴, R⁵, R⁶ and R⁷ independently of one another are each hydrogen or a hydrocarbon radical.

6. The process as claimed in claim 1 wherein the alkenyl compounds are vinyl compounds.

7. The process as claimed in claim 5 wherein the alkenyl compounds (Ia) are vinyl ethers.

8. The process as claimed in claim 5 wherein the alkenyl compounds (Ib) are N-vinylamides or N-vinylheterocycles.

9. The process as claimed in claim 8 wherein the N-vinylamide is N-vinyl-ε-caprolactam.

10. The process as claimed in claim 9 wherein the N-vinyl-ε-caprolactam is kept at a temperature of 35° to 60° C.
